(19) United States
(12) Patent Application Publication
(10) Pub. No.: US 2020/0370053 A1
(43) Pub. Date: Nov. 26, 2020

(54) METHOD FOR ENABLING NONMICROCYSTIN-PRODUCING MICROALGAE TO PRODUCE MICROCYSTIN AND MICROCYSTIN-PRODUCING MICROALGAE OBTAINED

(71) Applicant: Qiang WANG, Wuhan (CN)

(72) Inventor: Qiang WANG, Wuhan (CN)

(73) Assignee: Qiang WANG, Wuhan (CN)

(21) Appl. No.: 16/510,875

(22) Filed: Jul. 13, 2019

Related U.S. Application Data
(63) Continuation of application No. PCT/CN2019/089098, filed on May 29, 2019.

(30) Foreign Application Priority Data
May 21, 2019 (CN) 201910423393.X

(51) Int. Cl. C12N 15/52; C07K 14/195; C12N 1/20; C12N 15/74

(52) U.S. Cl. CPC C12N 15/52; C07K 14/195; C12N 2510/00; C12N 15/74; C12N 1/20

(57) ABSTRACT

This invention provides, for the first time, a method for producing microcystin by nonmicrocystin-producing microalgae and a microcystin-producing strain obtained thereby. This is the first report in the art to engineer nonmicrocystin-producing microalgae into microcystin-producing microalgae. We have realized synthesizing microcystin from inorganic substances by biosynthesis. Although the microcystin content produced by the obtained strain is low, it is still a great advance, and we will carry out subsequent research to increase the production of microcystin.

### CROSS-REFERENCE TO RELATED APPLICATIONS

This application is a continuation of International Patent Application No. PCT/CN2019/089098 with a filing date of May 29, 2019, designating the United States, now pending, and further claims priority to Chinese Patent Application No. 201910423393.X with a filing date of May 21, 2019. The content of the aforementioned applications, including any intervening amendments thereto, are incorporated herein by reference.

### CROSS-REFERENCE TO RELATED APPLICATIONS

This application includes a Sequence Listing submitted electronically as a text file named US_SL_ST25.txt, created on May 20, 2019, with a size of 2,737 bytes. The Sequence Listing is incorporated by reference herein.

### TECHNICAL FIELD

The present invention relates to the field of microalgae molecular biology, and more particularly to a method for enabling nonmicrocystin-producing microalgae to produce microcystin; and to the microcystin-producing microalgae obtained by the method.

### BACKGROUD OF THE PRESENT INVENTION

Microcystis is the most widespread, productive and harmful species among water-blooming cyanobacteria. Microcystis will release a large amount of microcystins after aging, death and dissolution, which endangers not only the ecology of water, but also the safety of drinking water for human.

Microcystin is a cyclic heptapeptide compound. It has been shown that microcystins can be enriched in hepatocytes, causing liver damage and even liver cancer.

Therefore, the research on microcystins has both ecological and medical significance. A gene cluster involved in the synthesis of microcystin has been identified from the microcystis genome. However, all of the microcystins used for research were extracted from the microcystin-producing microcystis so far. Although some researchers have attempted to introduce said gene cluster into a nonmicrocystin-producing microalgae, there has been none report on obtaining transgenic microcystin-producing microalgae based on non- microcystin-producing microalgae. The reasons may be multi-faceted. First, the gene cluster contains 10 genes with a length of 50-60 kb. It is difficult to introduce such a huge DNA into other microalgae so as to obtain a mutant strain and to express this gene. Secondly, even if a mutant strain capable of expressing said gene cluster is obtained, other substances necessary for synthesizing microcystin may not be contained in the environment of the microalgae.

Therefore, the construction of microalgae mutants capable of expressing microcystins requires both the identification of specific algae strains, substrates and other substances necessary for the synthesis of microcystins, and the development of a method for expressing of a gene cluster involved in the microcystins synthesis in the algae strain.

### SUMMARY OF PRESENT INVENTION

In order to solve the above problems, the present invention provides a method for producing microcystin by nonmicrocystin-producing microalgae, which comprises the step of expressing each gene in the mcy gene cluster in the nonmicrocystin-producing microalgae.

In a specific embodiment, the sequence of the mcy gene cluster can be obtained from GenBank No: AF183408.1.

In a specific embodiment, the nonmicrocystin-producing microalgae are Synechococcus. In a specific embodiment, the method comprises the steps of:

S1: introducing an expression cassette of each gene in the mcy gene cluster into the Synechococcus, and obtaining mcy Synechococcus;

S2: cultivating the mcy Synechococcus, and harvesting mcy Synechococcus algae cells; and

S3: extracting microcystin from the mcy Synechococcus algae cells.

In a specific embodiment, in S1, the expression cassette of each gene in the mcy gene cluster is introduced into the Synechococcus by a plasmid vector.

In a specific embodiment, S1 comprises the following steps:

S11: inserting the expression cassette of each gene in the mcy gene cluster into a plasmid vector to obtain an mcy gene expression vector; and

S12: introducing the mcy gene expression vector into the Synechococcus.

In a specific embodiment, the expression cassette of each gene in the mcy gene cluster consists of two operons.

In a specific embodiment, the expressions of the two operons are driven by two psbA2 promoters, respectively.

In a specific embodiment, the two psbA2 promoters are arranged back to back.

In a specific embodiment, an Ω fragment is arranged between the two psbA2 promoters.

In a specific embodiment, the sequence of the psbA2 promoter is set forth in SEQ ID NO: 1.

In a specific embodiment, in S11, the plasmid vector is a pGF plasmid into which replication origin for the replication of the plasmid vector in Synechococcus sp. PCC7942 is inserted.

In a specific embodiment, the sequence of the replication origin is set forth in SEQ ID NO: 2.

In a specific embodiment, the Synechococcus is Synechococcus sp. PCC7942.

The present invention further provides microalgae which can produce microcystin obtained by the above method.

The invention provides a method for producing microcystin by nonmicrocystin-producing microalgae, and further provides microcystin-producing microalgae obtained by the method. This is the first report in the art to engineer nonmicrocystin-producing microalgae into microcystin-producing microalgae. We have realized synthesizing microcystin from inorganic substances by biosynthesis. Although the microcystin content produced by the obtained strain is low, it is still a great advance, and we will carry out subsequent research to increase the production of microcystin based on the microalgae strain obtained.

### DESCRIPTION OF THE DRAWINGS

FIG. 1 is a schematic view showing the structure of a bidirectional promoter;

FIG. 2 is a map of the pGF plasmid;

FIG. 3 is a photograph of a plate after the pGF plasmid-transformed Synechococcus sp. PCC7942 plated, and it can be seen that no transformant grows on the plate;

FIG. 4 is a schematic diagram showing the structure of the mcy gene cluster in GenBank;

FIG. 5 is a flow chart for reconstructing an assembled mcy gene cluster;

FIG. 6 is a schematic view of a bidirectional promoter with a 5′ end of mcyA in one side and a 5′ end of mcyD in the other side;

FIG. 7 is a plasmid map of the expression vector mcy-pGF-ori;

FIG. 8 shows the expression of each gene of the mcy gene cluster in the transformants;

FIG. 9 is a comparison of the growth curves of mcy7942 and wt 7942 in BG11;

FIG. 10 is a HPLC-MS for mcy7942.

### DETAILED DESCRIPTION OF PREFERRED EMBODIMENTS

The principles and features of the present invention are described in the following with reference to the accompanying drawings. The following examples are for illustrative purpose only and are not intended to limit the scope of the invention.

1. Construction of a Bidirectional Promoter

First, we validated whether the bidirectional promoter in the myc gene cluster can initiate transcription in Synechococcus sp. PCC7942. The GFP gene was added to the 3′ and 5′ ends of this bidirectional promoter, respectively, and then ligated to the neutral platform of Synechococcus sp. PCC7942, and finally GFP fluorescence was observed under a microscope. The results showed that no GFP fluorescence was produced whether the GFP gene was added at the 3′ or 5′ end. Therefore, we inferred that this bidirectional promoter could not function in Synechococcus sp. PCC7942.

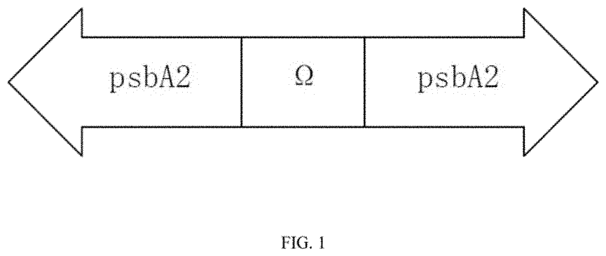

In order to express the gene cluster involved in microcystin synthesis in Synechococcus sp. PCC7942, we identified a strong promoter psbA2 (SEQ IT) NO: 1) which can be expressed in Synechococcus sp. PCC7942 by multiple validations, and built a bidirectional promoter based on this promoter. As shown in FIG. 1, the two psbA2 promoters were joined back-to-back with an Ω fragment (approximately 2 kb fragment obtained by dilation of pRL57 by Dral), ie, an Ω fragment in the middle, a psbA2 (SEQ ID) NO: 1) at each end, and the directions of both psbA2 are all facing away from Ω. This structure has two back-oriented psbA2 promoters on one fragment, enabling the promoters to initiate transcription of the respective operons on one plasmid. On the other hand, the Ω fragment can eliminate the interaction of the tertiary structures on both sides of the fragment. It also provides resistance to spectinomycin as a screening marker. In the present experiment, the construction process of the bidirectional promoter is done by the method of fusion PCR. However, the technical solution of the present invention is not limited thereto, and those skilled in the art can also perform the enzyme digestion or other construction methods.

2. Engineering of Expression Plasmid Vector

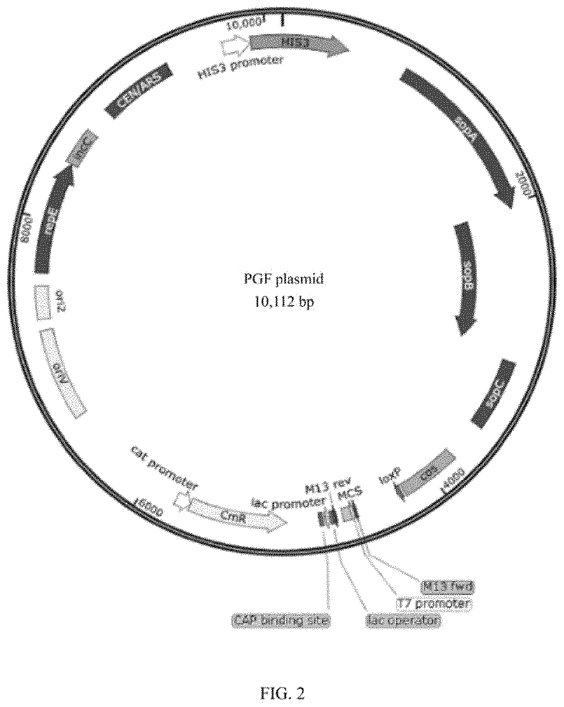

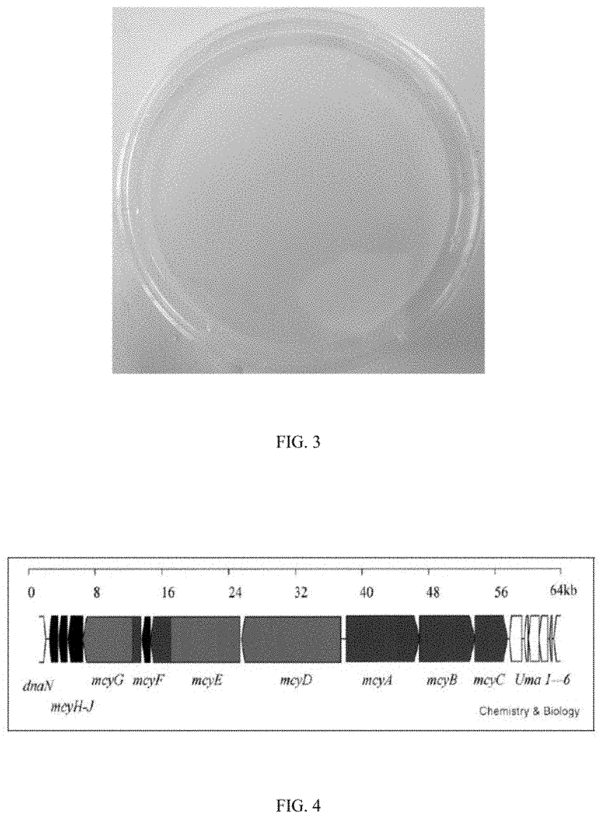

The pGF plasmid vector is a yeast-bacterial shuttle plasmid which is generally used for constructing an expression plasmid for protein expression in yeast, and its structure is shown in FIG. 2. However, we found in preliminary experiments that pGF was introduced into Synechococcus sp. PCC7942 and no positive clone was observed by corresponding resistance screening (FIG. 3). This indicates that pGF cannot be replicated in Synechococcus sp. PCC7942. In order to overcome this problem, we identified a replication initiation sequence (SEQ ID NO: 2), which was added to the BamHI site of the pGF vector to obtain a pGF-ori plasmid vector. After detection, pGF-ori can be replicated in Synechococcus sp. PCC7942. By combining the engineered plasmid pGF-ori with the bidirectional promoter constructed, we can construct a plasmid capable of constructing a clone in E. coli and expressing the gene in Synechococcus sp. PCC7942.

3. Synthesis of mcy Gene Cluster

The mcy gene cluster sequence can be obtained from GenBank, with accession number AF183408.1, involving 10 genes, in which mcyA, B, C are non-ribosomal peptide synthetase; mcyD is type I polyacetyl synthase; mcyG and E both have non-ribosomal peptide synthetase domain and type I polyacetyl synthase domain; mcyJ is an O-methylase; mcyF is a racemase; mcyl is a dehydrogenase, and mcyH is an ATP-dependent transmembrane transporter. As shown in FIG. 4, mcyA-C constitute an operon and mcyD-J constitute another operon. Since the length of the gene cluster is too long, it is impossible to amplify the entire gene cluster by one PCR. Therefore, referring to FIG. 5, we use the following steps to synthesize the gene cluster.

3.1 Extension of the 3′ End of the Bidirectional Promoter

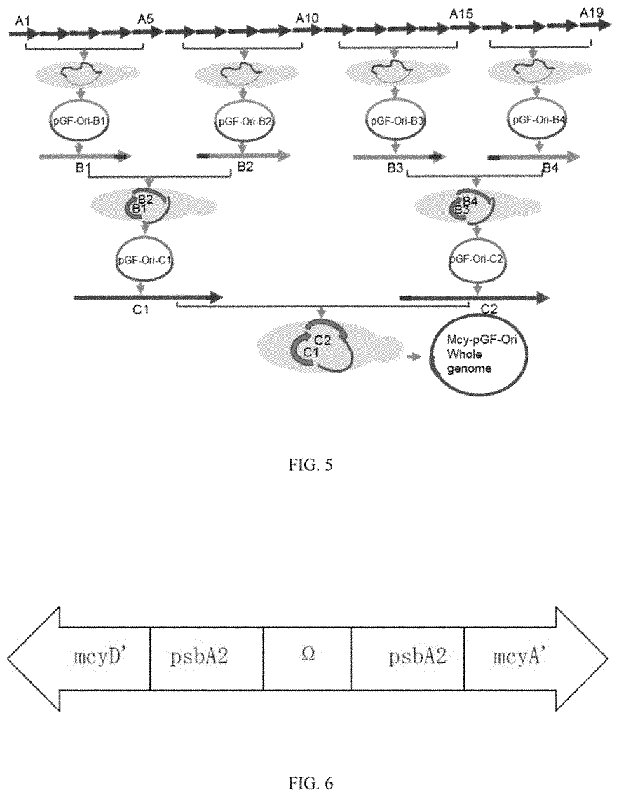

In order to facilitate the synthesis of the mcy gene cluster, in the construction of a bidirectional promoter, the about 80 bp sequences at the 5′ end of mcyA and mcyD were connected to the 3′ end of the two psbA2, respectively, and the structure of the bidirectional promoter with gene fragment thus formed is shown in FIG. 6.

3.2 Cloning A-level Fragments

Using the microcystis genome as a template, primers were designed to amplify the DNA fragment. The mcy gene cluster (with the promoter region removed) was segmented into fragments of about 3K for amplification, with an overlap region of 80 bp between each two adjacent fragments (with the promoter region deleted). These fragments are called A-level fragments. By this method, a total of 18 A-level fragments were amplified, and adding up the bidirectional promoter with an overlapping region of mcyA and mcyD, there is a total of 19 A-level fragments.

The A-level fragments were separately cloned into the T vector and confirmed by sequencing, and then digested and recovered for use.

3.3 Assembling of gene clusters

1) Preparation of yeast protoplasts

50 ml YEPD medium was added to a 250 ml Erlenmeyer flask. The single cell population VL6-48 was inoculated, and incubated at 30° C. under 220 rpm overnight (14-16h) to ensure ventilation, until the number of cells reaches about 2* 10⁷ cells/ml.

The yeast culture was washed once with sterile water, then resuspended in 1 M sorbitol solution, placed at 4° C. for at least 4 h, and centrifuged to remove the supernatant. The yeast cells were resuspended in 20 ml SPE solution, and 25 μl garcinia bacillus enzyme solution was added. 50 μl ME (mercaptoethanol) was added and mixed, incubated at 30° C. for 90 min, and gently shaken to destroy the cell wall.

Protoplasts were collected by low speed centrifugation, washed twice with 50 ml of 1 M sorbitol solution, and resuspended in 2 ml of STC solution.

2) Fragment assembly

The protoplasts were transformed with the A-level fragment and the plasmid vector pGF-ori. 200 ng A-level fragment was added to 200 82 l protoplast suspension, and one ligation reaction system contained the vector (400 ng) and five adjacent A-level fragments. 800 μl PEG 8000 solution was added to each centrifuge tube, mixed upside down, and incubated for 10 min at room temperature. The mixture was centrifuged at 590 g for 5 min at 5° C., and the supernatant was discarded. 800 μl SOS solution was added in each tube and gently resuspended, incubated at 30° C. for 40 min, with no vibration. The protoplast transformants obtained by the above transformation reaction were added to a medium containing 15 ml of dissolved SORB-TOP-His, gently mixed, and rapidly poured onto a selection medium of SORB-His. The plate was placed at 30° C. for 2-3 days. The grown transformants were detected correctly. Then, the B-level fragments in which adjacent fragments were ligated together were obtained by enzyme digestion. The above steps obtained 4 B-level fragments.

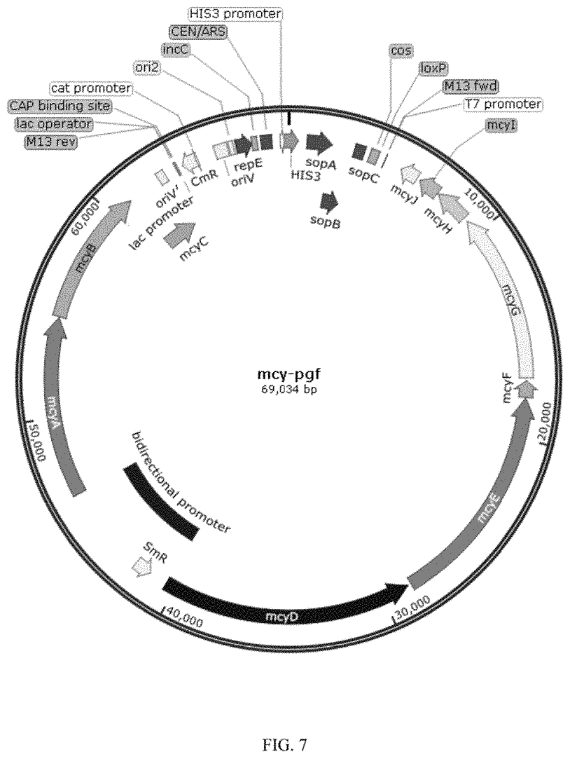

The four B-level fragments were assembled into two C-level fragments in the same manner, and the two C-level fragments were spliced together in the same manner, and attached onto a pGF vector so as to obtain an expression vector mcy-pGF-ori, with the plasmid map shown in FIG. 7.

4. Transformation of Synechococcus sp. PCC7942

The expression vector mcy-pGF-ori was transferred into the Synechococcus sp. PCC7942 cells by triple-strand binding transfer, and RP4 was used as a helper plasmid. The steps are provided as follows.

The mcy-pGF-ori-containing Escherichia coli and the RP4-containing Escherichia coli were cultured under viberation overnight. 250 μl of each strain was inoculated into 10 ml of non-resistant medium and shaken for 2-3 h to obtain a culture in the next day. The obtained culture was centrifuged separately, and the supernatant was discarded. The two bacteria were mixed and resuspended in 1 ml LB medium, and the supernatant was discarded. 200 μLB medium was resuspended, incubated at 30° C. for 1 h. 800 μl 7942 with OD at 0.8-1.0 was added, mixed, centrifuged, and supernatant discarded. The mixture was resuspended in 30 μl fresh BG11, plated on non-resistant BG11 plate containing 5% LB, and transferred to resistant plates over 18-24 h.

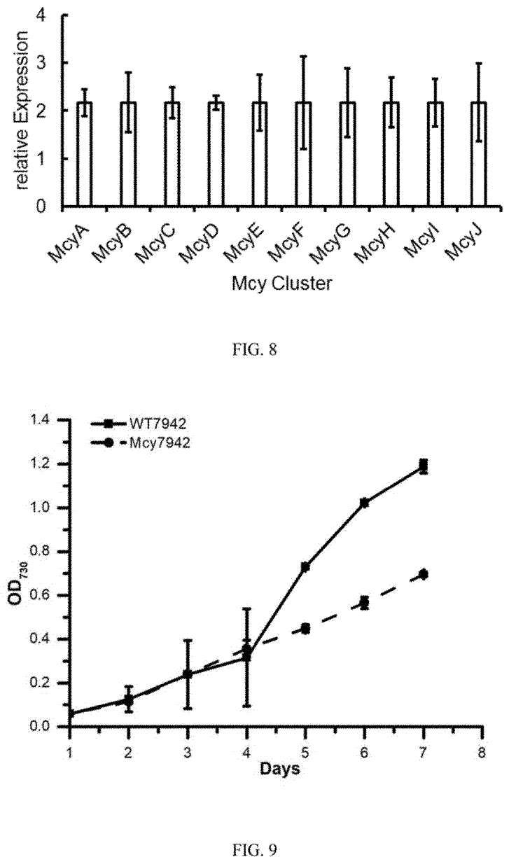

Positive transformants were screened after transformation. A plurality of transformants were selected for culture. qPCR showed that each gene in the mcy gene cluster was efficiently expressed in the transformants (FIG. 8).

5. mcy7942 Culture and Microcystin Detection

Mcy7942 was cultured using BG11 medium, and OD₇₃₀ was measured every day. The results are shown in FIG. 9. The growth rate of mcy7942 in the first four days was similar to that of wild type 7942. From day 5, mcy7942 grew slower than wild type 7942. The reason may lie in that mcy7942 began to synthesize microcystin from around the fourth day.

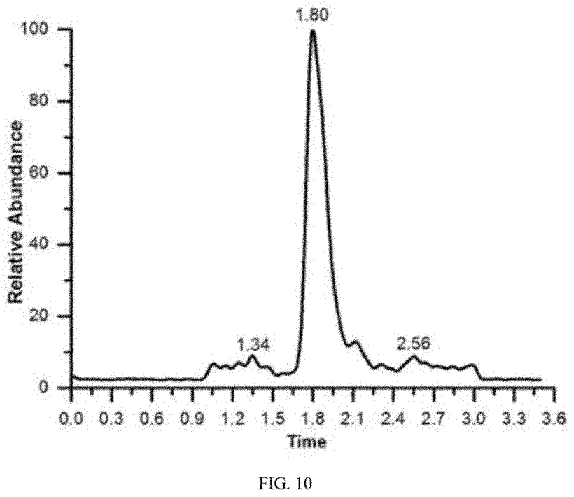

After the plateau period, the algal cells were collected. The microcystin content was detected by liquid chromatography-mass spectrometry, and the microcystis WT7806 and WT7942 were compared. The results showed that the microcystin content of WT7942 was 0, the microcystin content of mcy7942 was 78.9144 ng/g (DCW) (FIG. 10), and the microcystin content of microcystis WT7806 was 430595.2 ng/g (DCW).

We genetically engineered, for the first time, a nonmicrocystin-producing microalgae into a microcystin-producing microalgae, although the microcystin production of mcy7942 was relatively low. We performed similar experiments on other microalgae such as Synechocystis PCC6803, and found that they could not produce microcystin. It is speculated that the intracellar environment of Synechococcus sp. PCC7942 contains substrate or other substance necessary for the microcystin synthesis, while some other microalgae do not contain such substrate or substance.

The description above is only preferred embodiments of the present invention, and they are not intended to limit the scope of the present invention. Any modifications, equivalents, improvements and the like, which are include in the spirit and scope of the present invention, should fall into the protection scope of the present invention.

We claim:

1. A method for enabling nonmicrocystin-producing microalgae to produce microcystin, comprising a step of expressing each gene in the mcy gene cluster in nonmicrocystin-producing microalgae.

2. The method according to claim 1, wherein the nonmicrocystin-producing microalgae is Synechococcus.

3. The method according to claim 2, comprising the following steps:
S1: introducing an expression cassette of each gene in the mcy gene cluster into Synechococcus, to obtain mcy Synechococcus;
S2: cultivating the mcy Synechococcus, and harvesting mcy Synechococcus algae cells; and
S3: extracting microcystin from the Synechococcus algae cells.

4. The method according to claim 3, wherein in S1, the expression cassette of each gene in the mcy gene cluster is introduced into the Synechococcus by a plasmid vector.

5. The method according to claim 4, wherein S1 comprises steps of:
S11: inserting the expression cassette of each gene in the mcy gene cluster into the plasmid vector, to obtain an mcy gene expression vector; and
S12: introducing the mcy gene expression vector into the Synechococcus.

6. The method according to claim 5, wherein the expression cassette of each gene in the mcy gene cluster together constitute two operons.

7. The method according to claim 6, wherein expressions of the two operons are driven by two psbA2 promoters, respectively.

8. The method according to claim 7, wherein the sequence of the psbA2 promoter is set forth in SEQ ID NO: 1.

9. The method according to claim 5, wherein in S11, the plasmid vector is a pGF plasmid into which a replication origin for the plasmid vector to replicate in the Synechococcus is inserted.

10. A microcystin-producing microalgae, which is obtained by the method according to claim 2.


SEQUENCE LISTING (2 sequences)

SEQ ID NO: 1 - DNA, 503 bp, Artificial Sequence
  Designed noncoding sequence acting as a promoter
tgcggctttagcgttccagtggatatttgctgggggttaatgaaacattgtggcggaacccagggacaatgtgaccaaaaaattcagggatatcaataagtattaggtatatggatcataattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataacc

SEQ ID NO: 2 - DNA, 1022 bp, Artificial Sequence
  Designed noncoding sequence acting as a replication initiator
ttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagca